(19) United States
(12) Patent Application Publication
(10) Pub. No.: US 2025/0332358 A1
(43) Pub. Date: Oct. 30, 2025

(54) ATOMIZING MODULE AND DRUG DELIVERY DEVICE INCLUDING THE SAME

(71) Applicant: HCMed Innovations Co., LTD., Taipei City (TW)

(72) Inventor: HSIN-YI PAI, New Taipei City (TW)

(21) Appl. No.: 18/983,377

(22) Filed: Dec. 17, 2024

Related U.S. Application Data
(60) Provisional application No. 63/639,866, filed on Apr. 29, 2024.

(30) Foreign Application Priority Data
Jul. 31, 2024 (CN) 202411036277.X

(51) Int. Cl. A61M 11/00

(52) U.S. Cl. CPC A61M 11/001

(57) ABSTRACT

An atomizing module and a drug delivery device including the same are provided. The atomizing module includes a ring element, a piezoelectric sheet, and a porous element. The ring element includes a central region and an outer region surrounding the central region. The piezoelectric sheet is arranged at the outer region of the ring element. The piezoelectric sheet includes a first electrode and a second electrode, and the first electrode and the second electrode are respectively arranged on different sides of the piezoelectric sheet. The porous element is arranged at the central region of the ring element. The first electrode of the piezoelectric sheet faces towards the ring element, the ring element has a first lead that is integrally formed, and the second electrode of the piezoelectric sheet is electrically connected to a second lead through a joint element.

### CROSS-REFERENCE TO RELATED PATENT APPLICATION

This application claims the benefit of priority to the U.S. Provisional Patent Application Ser. No. 63/639,866, filed on Apr. 29, 2024, which application is incorporated herein by reference in its entirety.

This application claims the benefit of priority to China Patent Application No. 202411036277.X, filed on Jul. 31, 2024, in the People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an atomizing module and a drug delivery device including the same, and more particularly to an atomizing module and a drug delivery device including the same used for atomizing liquid medicine.

### BACKGROUND OF THE DISCLOSURE

Nebulized drug delivery is to atomize the liquid medicine, allowing the user to inhale the atomized liquid medicine into the lungs to achieve direct and rapid therapeutic effects. Conventional vibrating atomizer devices use the high-frequency vibration of the piezoelectric sheet to convert the liquid medicine into tiny aerosol molecules, thereby providing inhaled drug delivery treatment.

In order for the atomization device to be easily carried by the user, it is necessary to further reduce the size of the atomization device. Therefore, reducing the size of the atomizing module in a micron atomization device has become an important issue.

When it is desired to have the piezoelectric sheet of the aforementioned atomizing module be electrically conducted in a limited space, one solution is having one of the positive and negative electrodes of the piezoelectric sheet be placed on the same side through extension and guidance, and be connected to the driving module, respectively. Therefore, the electrode can extend to the other side by coating silver glue on the piezoelectric sheet and bending the silver glue from one side to the other along the surface of the piezoelectric sheet. However, the aforementioned method may cause wire breakage during the stretching and bending process due to uneven coating of the silver glue, which can lead to the piezoelectric sheet being unable to be conducted.

Therefore, how to simplify the conduction steps of piezoelectric sheet through structural design improvements has become one of the important issues to be solved in the related art.

### SUMMARY OF THE DISCLOSURE

In response to the above-referenced technical inadequacies, the present disclosure provides an atomizing module and a drug delivery device including the same.

In order to solve the above-mentioned problems, one of the technical aspects adopted by the present disclosure is to provide an atomizing module, including: a ring element having a central region and an outer region surrounding the central region; a piezoelectric sheet arranged at the outer region of the ring element, the piezoelectric sheet including a first electrode and a second electrode, and the first electrode and the second electrode being respectively arranged on different sides of the piezoelectric sheet; and a porous element arranged at the central region of the ring element. The first electrode of the piezoelectric sheet faces towards the ring element, the ring element has a first lead that is integrally formed, and the second electrode of the piezoelectric sheet is electrically connected to a second lead through a joint element.

In one of the possible or preferred embodiments, the first lead extends from the outer region of the ring element in a direction away from the ring element.

In one of the possible or preferred embodiments, the first lead extends from the outer region of the ring element along at least two directions.

In one of the possible or preferred embodiments, the first electrode is a positive electrode, the second electrode is a negative electrode, the first lead is a positive lead, and the second lead is a negative lead.

In one of the possible or preferred embodiments, the ring element and the piezoelectric sheet are bonded with an adhesive layer.

In one of the possible or preferred embodiments, the first electrode of the piezoelectric sheet is electrically connected to the ring element through a conductive glue.

In one of the possible or preferred embodiments, the adhesive layer is a conductive adhesive.

In one of the possible or preferred embodiments, the joint element is a solder.

In order to solve the above-mentioned problems, another one of the technical aspects adopted by the present disclosure is to provide a drug delivery device, including: a host; a medicine cup connected with the host and including a nozzle; and the aforementioned atomizing module being arranged in the medicine cup.

In one of the possible or preferred embodiments, the nozzle includes a first electrode post and a second electrode post, one end of the first lead has a first ring to be sleeved on the first electrode post, and one end of the second lead has a second ring to be sleeved on the second electrode post.

In one of the possible or preferred embodiments, the atomizing module is arranged perpendicular to an outlet direction of the nozzle.

In one of the possible or preferred embodiments, a spray port of the atomizing module is perpendicular to an outlet direction of the nozzle.

In one of the possible or preferred embodiments, the drug delivery device further includes a detection module, the detection module is disposed in the host and is used to cooperate with the ring element to detect a remaining amount of liquid medicine in the medicine cup.

Therefore, in the atomizing module and the drug delivery device including the same provided by the present disclosure, by virtue of “the ring element has a first lead that is integrally formed,” and “the second electrode of the piezoelectric sheet is electrically connected to a second lead through a joint element,” the process steps of the atomizing module can be simplified and the risk of the piezoelectric film being unable to be driven due to broken silver glue in the existing technology can be avoided.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a front schematic view of an atomizing module of the present disclosure.

FIG. 2 is a perspective view of the atomizing module of the present disclosure.

FIG. 3 is a schematic planar view along a direction III-III of FIG. 2.

FIG. 4 is a rear schematic view of the atomizing module of the present disclosure.

FIG. 5 is a perspective view of a drug delivery device of the present disclosure.

FIG. 6 is an exploded view of the atomizing module being installed in the drug delivery device of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

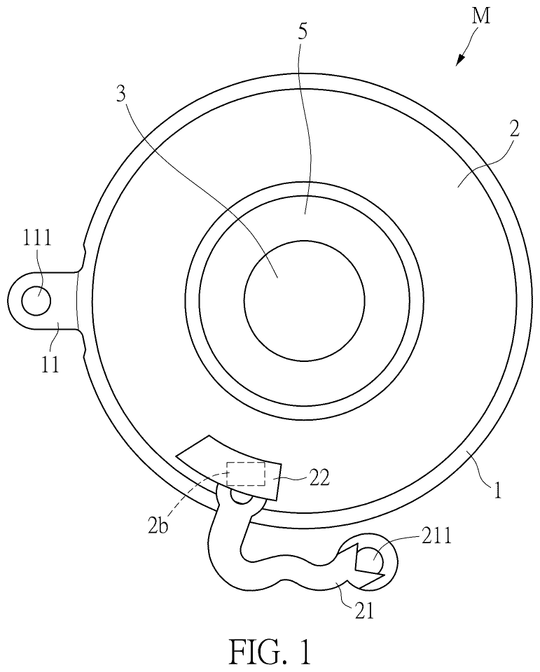

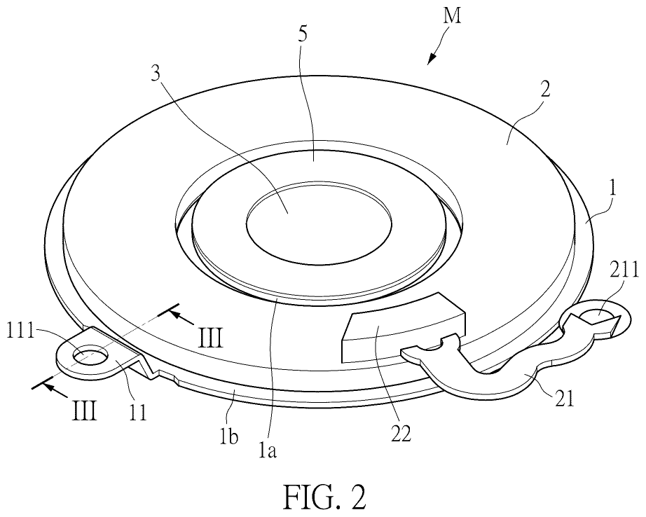

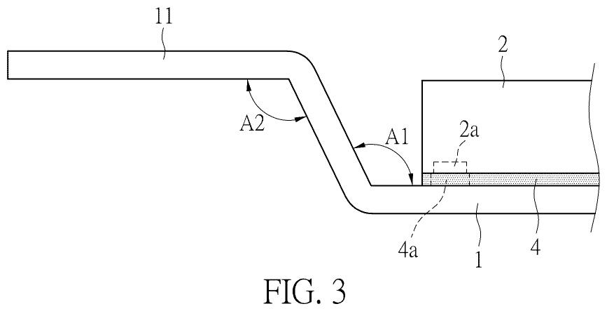

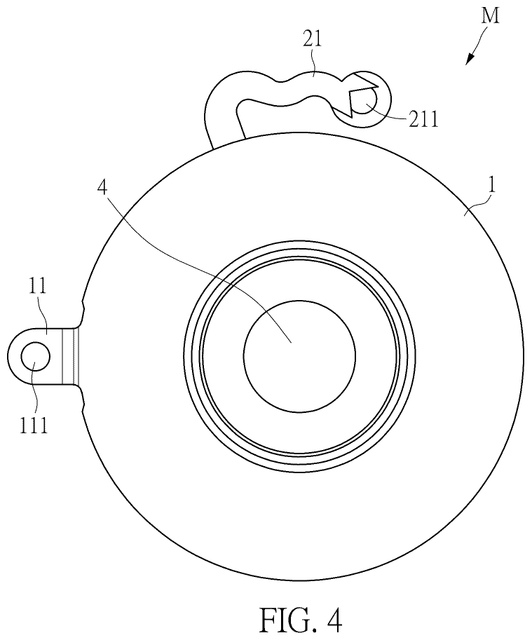

Referring to FIG. 1 to FIG. 4, FIG. 1 is a front schematic view of an atomizing module of the present disclosure, FIG. 2 is a perspective view of the atomizing module of the present disclosure, FIG. 3 is a schematic planar view along a direction III-III of FIG. 2, and FIG. 4 is a rear schematic view of the atomizing module of the present disclosure. The present disclosure provides an atomizing module M, including: a ring element 1, a piezoelectric sheet 2, and a porous element 3. The ring element 1 is substantially circular and can be used to carry the piezoelectric sheet 2. The ring element 1 includes a central region la and an outer region 1b surrounding the central region la. In other words, the piezoelectric sheet 2 is disposed on the outer region 1b of the ring element 1, and the piezoelectric sheet 2 can be fixed on the ring element 1 through an adhesive layer 4. The piezoelectric sheet 2 includes a first electrode 2a and a second electrode 2b, and the first electrode 2a and the second electrode 2b are respectively arranged on different sides of the piezoelectric sheet 2. The porous element 3 is arranged at the central region la of the ring element 1. The first electrode 2a of the piezoelectric sheet 2 is faced to the ring element 1. The ring element 1 has a first lead 11 that is integrally formed, the second electrode 2b of the piezoelectric sheet 2 is electrically connected to a second lead 21 through a joint element 22.

Since the ring element 1 has the first lead 11 integrally formed, the first lead 11 does not need to be combined with the ring element 1 through welding as in the related art, thereby achieving the purpose of streamlining the manufacturing process. In addition, the first lead 11 may extend from the outer region 1b of the ring element 1 in a direction away from the ring element 1, or may extend from the outer region 1b of the ring element 1 along at least two directions.

For example, in one embodiment, the first lead 11 integrally formed with the ring element 1 may extend from a side of the first electrode 2a of the piezoelectric sheet 2 in a direction away from the ring element 1 to a side of the second electrode 2b of the piezoelectric sheet 2.

Optionally, in other embodiments, as shown in FIG. 2 and FIG. 3, the first lead 11 may be extended in the horizontal direction away from the ring element 1 along the outer region 1b of the ring element 1, and then the first lead 11 may be bent in the vertical direction at a first degree A1 and continue to extend. Finally, the first lead 11 may be bent at a second degree A2 in the horizontal direction, and then continue to extend in a direction away from the ring element 1.

Therefore, the abovementioned two extension manners of the first lead 11 can achieve the purpose of extending the first lead 11 to the same side as the second lead 21, thereby eliminating the need to bend the silver glue in the related art, and avoid the risk of line interruption during the bending process of silver glue.

In addition, during the process of electrically connecting the piezoelectric sheet 2 to the second lead 21 through the joint element 22, the joint element 22 is preferably a solder, but is not limited thereto. In addition, the position where the joint element 22 is disposed on the piezoelectric sheet 2 is the second electrode 2b of the piezoelectric sheet 2.

Specifically, the ring element 1 and the piezoelectric sheet 2 may have an annular hollow shape to place the porous element 3 in the hollow position. In one embodiment, the porous element 3 can be fixed at the center of the ring element 1 through the fixed member 5. For example, the porous element 3 may be a mesh, and the fixed member 5, the porous element 3 and the piezoelectric sheet 2 are arranged on the same side of the ring element 1. However, the present disclosure is not limited to the aforementioned examples.

When the liquid medicine is supplied to the atomizing module M, and the driving voltage is supplied to the atomizing module M through the first lead 11 and the second lead 21, the piezoelectric sheet 2 will generate high-frequency oscillation, thereby actuating the porous element 3, so that the porous element 3 may atomize the liquid medicine to generate aerosol molecules. In other words, the liquid medicine may pass through the multiple through holes of the porous element 3 and become an aerosol to achieve the purpose of atomized drug administration. Specifically, the porous element 3 may be made of palladium-nickel alloy or ceramic. However, the present disclosure is not limited to the aforementioned examples.

In one aspect of the present disclosure, the first electrode 2a is a positive electrode, the second electrode 2b is a negative electrode, the first lead 11 may be a positive electrode lead, and the second lead 21 may be a negative electrode lead. However, the aforementioned details are disclosed for exemplary purposes only, and are not meant to limit the scope of the present disclosure. In another aspect of the present disclosure, the first electrode 2a may be the negative electrode, the second electrode 2b may be the positive electrode, the first lead 11 may be the negative electrode lead, and the second lead 21 may be the positive electrode lead.

In one embodiment of the present disclosure, when the piezoelectric sheet 2 is fixed on the ring element 1 through the adhesive layer 4, and the adhesive layer 4 is a non-conductive adhesive, the first electrode 2a of the piezoelectric sheet 2 is electrically connected to the ring element 1 through a conductive glue 4a. The conductive glue 4a may also be silver glue or other forms. In addition, the conductive adhesive 4a may further extend along the surface of the first lead 11 in a direction away from the ring component 1, and be electrically connected to the following electrode post.

In another embodiment of the present disclosure, the adhesive layer 4 may directly be in the form of conductive adhesive, so that the first electrode 2a of the piezoelectric sheet 2 is directly in contact with the conductive adhesive layer 4 and is electrically connected to the entire ring element 1. In other words, when the first electrode 2a of the piezoelectric sheet 2 is the positive electrode, through the conduction of the conductive adhesive layer 4, the entire ring element 1 will be used as the positive electrode, and the first lead 11 will be used as the positive electrode.

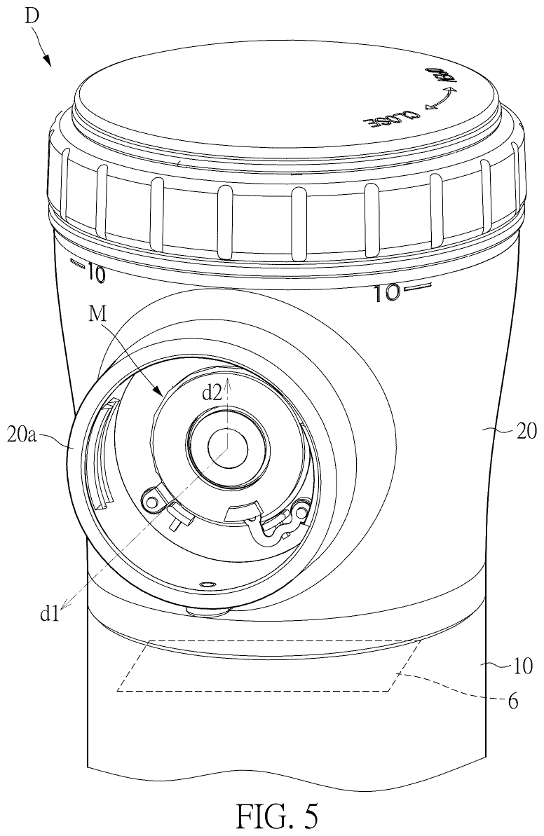

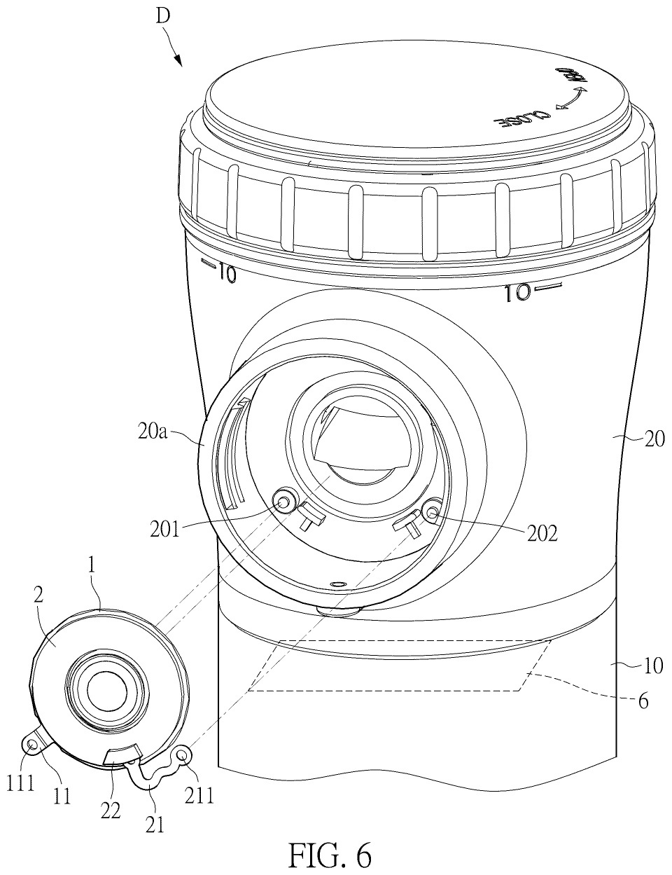

Referring to FIG. 5 and FIG. 6, FIG. 5 is a perspective view of a drug delivery device of the present disclosure, and FIG. 6 is an exploded view of the atomizing module being installed in the drug delivery device of the present disclosure. The present disclosure also provides a drug delivery device D, including: a host 10 and a medicine cup 20. The medicine cup 20 is connected with the host 10. For example, the medicine cup 20 is connected above the host 10. The host 10 may be used to provide power to the medicine cup 20. The medicine cup 20 may include a nozzle 20a, and a first electrode post 201 and a second electrode post 202 are provided at the position of the nozzle 20a corresponding to the atomizing module M.

In addition, the atomizing module M of the present disclosure may arrange in the medicine cup 20. Moreover, the first lead 11 of the atomizing module M may include a first ring 111, the second lead 21 of the atomizing module M may include a second ring 211. Specifically, the first ring 111 may be disposed at an end of the first lead 11 away from the ring element 1, and the second ring 211 may be disposed at an end of the second lead 21 away from the piezoelectric sheet 2.

Accordingly, when the atomizing module M is arranged in the medicine cup 20, the first ring 111 of the first lead 11 may be sleeved on the first electrode post 201, and the second ring 211 of the second lead 21 may be sleeved on the second electrode post 202, thereby achieving the purpose of electrical connection.

Referring to FIG. 5, in one embodiment of the present disclosure, taking the direction d1 as the direction parallel to the horizontal plane and the direction d2 as the direction perpendicular to the horizontal plane as an example, the atomizing module M may be installed in the medicine cup 20 perpendicular to the direction d1. Specifically, the atomizing module M may be set perpendicular to the outlet direction of the nozzle. Optionally, a spray port of the atomizing module M may be perpendicular to the outlet direction of the nozzle 20a (i.e. The atomizing module M is arranged perpendicular to the direction d2). However, the present disclosure is not limited to the aforementioned examples.

Referring to FIG. 5 and FIG. 6, the drug delivery device D of the present disclosure may further include a detection module 6, the detection module 6 is preferably arranged in the host 10. The detection module 6 may cooperate with the ring element 1 to detect the remaining amount of liquid medicine in the medicine cup 20. Specifically, in one embodiment, since the atomizing module M provided by the present disclosure may use the entire ring element 1 as the positive electrode, the electrical signal from the positive electrode post (i.e., the first electrode post 201) may be conducted to the medicine cup 20 through the ring element 1 in the liquid medicine in the medicine cup 20, and the detection module 6 may receive the voltage change from the liquid medicine in a non-contact manner to determine the remaining amount of the liquid medicine in the medicine cup 20.

### Beneficial Effects of the Embodiments

In conclusion, in the atomizing module and the drug delivery device including the same provided by the present disclosure, by virtue of “the ring element has a first lead that is integrally formed,” and “the second electrode of the piezoelectric sheet is electrically connected to a second lead through a joint element,” the process steps of the atomizing module can be simplifies and avoids the risk of the piezoelectric film being unable to be driven due to broken silver glue in the existing technology.

Moreover, since the atomizing module provided by the present disclosure may use the entire ring element as the positive electrode, the electrical signal from the positive electrode post may be conducted through the ring element to the liquid medicine in the medicine cup, and the detection module may be used to detect the atomizing module. The detection module receives the voltage change from the liquid drug in a non-contact manner to determine the remaining amount of the liquid drug in the medicine cup, so that the atomizing module in the present disclosure further includes the effect of liquid detection.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An atomizing module, comprising:
a ring element having a central region and an outer region surrounding the central region;
a piezoelectric sheet arranged at the outer region of the ring element, wherein the piezoelectric sheet includes a first electrode and a second electrode, and the first electrode and the second electrode are respectively arranged on different sides of the piezoelectric sheet; and
a porous element arranged at the central region of the ring element;
wherein the first electrode of the piezoelectric sheet faces towards the ring element, the ring element has a first lead that is integrally formed, and the second electrode of the piezoelectric sheet is electrically connected to a second lead through a joint element.

2. The atomizing module according to claim 1, wherein the first lead extends from the outer region of the ring element in a direction away from the ring element.

3. The atomizing module according to claim 1, wherein the first lead extends from the outer region of the ring element along at least two directions.

4. The atomizing module according to claim 1, wherein the first electrode is a positive electrode, the second electrode is a negative electrode, the first lead is a positive lead, and the second lead is a negative lead.

5. The atomizing module according to claim 1, wherein the ring element and the piezoelectric sheet are bonded with an adhesive layer.

6. The atomizing module according to claim 5, wherein the first electrode of the piezoelectric sheet is electrically connected to the ring element through a conductive glue.

7. The atomizing module according to claim 5, wherein the adhesive layer is a conductive adhesive.

8. The atomizing module according to claim 1, wherein the joint element is a solder.

9. A drug delivery device, comprising:
a host;
a medicine cup connected with the host and including a nozzle; and
the atomizing module as claimed in claim 1 arranged in the medicine cup.

10. The drug delivery device according to claim 9, wherein the nozzle includes a first electrode post and a second electrode post, one end of the first lead has a first ring to be sleeved on the first electrode post, and one end of the second lead has a second ring to be sleeved on the second electrode post.

11. The drug delivery device according to claim 9, wherein the atomizing module is arranged perpendicular to an outlet direction of the nozzle.

12. The drug delivery device according to claim 9, wherein a spray port of the atomizing module is perpendicular to an outlet direction of the nozzle.

13. The drug delivery device according to claim 9, further comprising a detection module, the detection module being disposed in the host and being used to cooperate with the ring element to detect the remaining amount of liquid medicine in the medicine cup.
